(19) United States
(12) Patent Application Publication
(10) Pub. No.: US 2023/0177684 A1
(43) Pub. Date: Jun. 8, 2023

(54) METHOD AND APPARATUS FOR EVALUATING INSPIRATION-LEVEL QUALITY OF CHEST RADIOGRAPHIC IMAGE

(71) Applicant: AJOU UNIVERSITY INDUSTRY-ACADEMIC COOPERATION FOUNDATION, Gyeonggi-do (KR)

(72) Inventors: Jung Won LEE, Seoul (KR); Yang Gon KIM, Gyeonggi-do (KR); Su Bin BAE, Gyeonggi-do (KR); Hyeon Woong JANG, Gyeonggi-do (KR)

(21) Appl. No.: 18/057,422

(22) Filed: Nov. 21, 2022

(30) Foreign Application Priority Data
Dec. 2, 2021 (KR) 10-2021-0171021

(51) Int. Cl. G06T 7/00; G06T 7/33; G06T 7/68; G06V 10/26

(52) U.S. Cl. CPC G06T 7/0012; G06T 7/33; G06T 7/68; G06V 10/26; G06T 2207/30012; G06T 2207/30061; G06V 2201/033; G06V 2201/031; G06T 2207/10116

(57) ABSTRACT

The present invention relates to a method and apparatus for evaluating inspiration-level quality of a chest radiographic image, wherein the method includes extracting a lung region from a chest radiographic image, detecting a rib cage from the extracted lung region, analyzing a degree of inspiration when the chest radiographic image is captured, and evaluating quality of the chest radiographic image. It is possible for the present invention to be applied to other embodiments.

### CROSS-REFERENCE TO RELATED APPLICATION

This application claims priority to and the benefit of Korean Patent Application No. 2021-0171021, filed on Dec. 2, 2021, the disclosure of which is incorporated herein by reference in its entirety.

### BACKGROUND

### 1. Field of the Invention

The present invention relates to a method and apparatus for evaluating inspiration-level quality of a chest radiographic image.

### 2. Discussion of Related Art

Medical imaging technologies are useful technologies that allow us to understand physical states of various organs in the human body. Commonly used medical imaging technologies include digital radiography (using X-rays), computed tomography (CT), and magnetic resonance imaging (MRI). Diagnosis methods using these imaging technologies have various advantages and disadvantages and points to be considered in applying the technologies.

In CT, MRI, or the like, although body organs can be shown in high-definition high-resolution images, it is difficult to say that CT, MRI, or the like is suitable for all patients because of the large amounts of radiation, complicated examination procedures, and high costs. On the other hand, a diagnostic method using radiography (using X-rays) is a very useful diagnostic method in which much medical information can be obtained through a very simple procedure.

Among X-rays, chest X-rays are basically used to identify various chest and heart-related diseases. In general, such X-rays are subjectively read by observers such as radiologists, clinicians, or the like. Therefore, there are deviations in accuracy of reading due to differences in observers' careers and experience, and misdiagnosis occurs frequently due to low quality of radiographic images. Accordingly, a demand for acquiring high-quality X-rays for more accurate X-ray reading is increasing.

### SUMMARY OF THE INVENTION

Embodiments of the present invention are directed to solving these conventional problems and providing a method and apparatus for evaluating inspiration-level quality of a chest radiographic image, which can determine the inspiration-level quality of the chest radiographic image.

According to an aspect of the present invention, there is provided a method of evaluating inspiration-level quality of a chest radiographic image, which includes extracting a lung region from a chest radiographic image, detecting a rib cage from the extracted lung region, analyzing a degree of inspiration when the chest radiographic image is captured, and evaluating quality of the chest radiographic image.

The evaluating of the quality may include evaluating the quality on the basis of a range in which lungs are included in the chest radiographic image identified from the extracted lung region, whether a patient's posture identified from the detected rib cage is aligned, and the degree of inspiration.

The evaluating of the quality may include checking that a 1^{st} rib, a lateral costal transverse sinus, and all ribs are included in the chest radiographic image to identify the range in which the lungs are included in the chest radiographic image.

The evaluating of the quality may include checking whether distances between a spinous process of a thoracic vertebra and inner ends of both clavicles are the same and positions of scapulae in the chest radiographic image to check whether the patient's posture is aligned.

The evaluating of the quality may include checking whether a contact point between a vertical line at a center of the clavicle and a right diaphragm in the chest radiographic image is lower than a lower edge of a posterior 10^{th} rib to check the degree of inspiration.

The evaluating of the quality may include analyzing positions of the ribs and checking the number of the ribs on the basis of brightness of pixels in a y-axis direction for the ribs crossing the vertical line among the ribs identified in the chest radiographic image to evaluate the quality.

According to another aspect of the present invention, there is provided an apparatus for evaluating inspiration-level quality of a chest radiographic image, which includes an input unit configured to generate a quality evaluation request signal for a chest radiographic image, and a control unit configured to extract a lung region from the chest radiographic image, detect a rib cage from the extracted lung region, and evaluate quality of the chest radiographic image on the basis of a degree of inspiration when the chest radiographic image is captured.

The control unit may evaluate the quality on the basis of a range in which lungs are included in the chest radiographic image identified from the extracted lung region, whether a patient's posture identified from the detected rib cage is aligned, and the degree of inspiration.

The control unit may check that a 1^{st} rib, a lateral costal transverse sinus, and all ribs are included in the chest radiographic image to identify the range in which the lungs are included in the chest radiographic image.

The control unit may check whether distances between a spinous process of a thoracic vertebra and inner ends of both clavicles are the same and positions of scapulae in the chest radiographic image to check whether the patient's posture is aligned.

The control unit may check whether a contact point between a vertical line at a center of the clavicle and a right diaphragm in the chest radiographic image is lower than a lower edge of a posterior 10^{th} rib to check the degree of inspiration.

The control unit may analyze positions of the ribs and checks the number of the ribs on the basis of brightness of pixels in a y-axis direction for the ribs crossing the vertical line among the ribs identified in the chest radiographic image to evaluate the quality.

The control unit may extract the lung region from the chest radiographic image using an object recognition algorithm.

The control unit may detect the rib cage from the lung region using an image binarization and noise removal algorithm.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 is a diagram illustrating a main configuration of an electronic device for evaluating inspiration-level quality of a chest radiographic image according to an embodiment of the present invention;

FIG. 2 is a flowchart for describing a method of evaluating inspiration-level quality of a chest radiographic image according to an embodiment of the present invention; and

FIGS. 3 to 5 are exemplary views of screens for describing the method of evaluating the inspiration-level quality of the chest radiographic image according to the embodiment of the present invention.

### DETAILED DESCRIPTION OF EXEMPLARY EMBODIMENTS

Hereinafter, exemplary embodiments of the present invention will be described in detail with reference to the accompanying drawings. The following detailed description together with the accompanying drawings is intended to describe the exemplary embodiments of the present invention, and is not intended to represent the only embodiments through which the present invention may be embodied. In the drawings, parts not related to the description may be omitted to clearly describe the present invention, and the same reference numerals may be used for the same or similar elements throughout the specification.

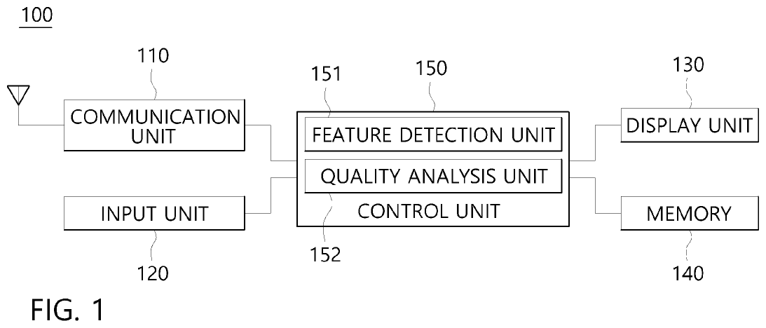

FIG. 1 is a diagram illustrating a main configuration of an electronic device for evaluating inspiration-level quality of a chest radiographic image according to an embodiment of the present invention.

Referring to FIG. 1, an electronic device 100 according to the present invention may include a communication unit 110, an input unit 120, a display unit 130, a memory 140, and a control unit 150. In addition, in the embodiment of the present invention, an example in which the electronic device 100 receives a chest radiographic image acquired from an external device (not illustrated), for example, an X-ray device, or receives a chest radiographic image stored in a server (not illustrated) to evaluate inspiration-level quality of the corresponding image is illustrated, but the present invention is not necessarily limited thereto. The electronic device 100 may be an X-ray device for acquiring a chest radiographic image, and in this case, the electronic device 100 may further include an image acquisition unit (not illustrated) capable of acquiring a chest radiographic image.

The communication unit 110 communicates with an X-ray device and a server (not illustrated) that stores a plurality of chest radiographic images. Accordingly, the communication unit 110 may perform wireless communication such as 5^{th} generation (5G) communication, Long-Term Evolution (LTE), LTE-advanced (LTE-A), Wi-Fi, or the like. In particular, the communication unit 110 may perform wired communication using cables or the like for communication with the X-ray device.

The input unit 120 generates input data in response to a user input of the electronic device 100. To this end, the input unit 120 may include an input device such as a keyboard, a mouse, a keypad, a dome switch, a touch panel, a touch key, a button, or the like.

The display unit 130 outputs output data according to an operation of the electronic device 100. To this end, the display unit 130 may include a display device such as a liquid crystal display (LCD), a light-emitting diode (LED) display, an organic LED (OLED) display, or the like. In addition, the display unit 130 may be implemented in the form of a touch screen in combination with the input unit 120.

An algorithm for extracting a lung region as a chest image from a chest radiographic image and detecting a rib cage from the chest image may be stored in the memory 140. Further, an algorithm for analyzing a degree of inspiration when the chest radiographic image is captured and evaluating inspiration-level quality of the chest image may be stored in the memory 140. As shown in Table 1 below, such algorithms may check an inclusion range, a patient's posture, and a degree of inspiration among guidelines that are standard for evaluating the inspiration-level quality of the chest image, and thus the inspiration-level quality of the chest image can be more accurately evaluated.

TABLE 1

| Information | Items | Evaluation content |
|---|---|---|
| Normal | Examination | Patient name, sex, age, patient record |
| | cover | number, photographing date, |
| | | photographing institution, photographer |
| | | information |
| | Examination | Extent to which cover covers chest |
| | cover position | inclusion range (including ribs) |
| | Position | Left and right display |
| | indication | |
| | Adequacy of | Aging (yellowing) of submitted image |
| | developing | |
| | conditions | |
| Image | Inclusion range | Upper: including 1^{st} rib, |
| | | Lower: lateral costal transverse sinus |
| | | (3 cm or more downward), Left and |
| | | right: including all ribs |
| | Patient's | Left-right symmetry: distances between |
| | posture | spinous process of thoracic vertebrae |
| | | and inner ends of both clavicles are the |
| | | same and positions of scapulae |
| | Degree of | Normal inspiration: contact point |
| | inspiration | between vertical line at center |
| | | of clavicle and right diaphragm is |
| | | lower than lower edge of posterior |
| | | 10^{th} rib |
| | Artificial | Artificial shading (due to patient's |
| | shading | clothes, attachments, hair, etc.) |
| | | from the outside, internal artificial |
| | | shading, artificial shading (e.g., |
| | | stains, scratches, fingerprints, |
| | | roller marks, static electricity, |
| | | grid artifacts, fog, photosensitivity, |
| | | etc.) of unknown cause, breathing, |
| | | or movement |
| | Transmission | Degree of observation of pulmonary |
| | state, resolution, | blood vessels, observation of |
| | and contrast | pulmonary blood vessels behind |
| | | the heart and descending aorta, |
| | | observation of sub-diaphragm vessels, |
| | | observation of costal margin, |
| | | observation of diaphragm, observation |
| | | of thoracic intervertebral disc space, |
| | | or observation of trachea and bronchi |

The control unit 150 extracts a lung region as a chest image from the chest radiographic image received from the X-ray device or the server using the algorithms stored in the memory 140, and detects a rib cage from the chest image. The control unit 150 analyzes a degree of inspiration when the chest radiographic image is captured, and evaluates inspiration-level quality of the chest image. The control unit 150 may display a result of the evaluation on the display unit 130 only when the inspiration-level quality of the chest image is greater than or equal to a threshold, or the control unit 150 may display a message indicating that accurate reading of the chest image is impossible when the inspiration-level quality of the chest image is less than the threshold.

To this end, the control unit 150 may include a feature detection unit 151 and a quality analysis unit 152. The feature detection unit 151 receives an image evaluation request signal for the chest radiographic image from the input unit 120, and calls the chest radiographic image from any one of the X-ray device, the server, and the memory 140 for image evaluation. The feature detection unit 151 may identify a contact point between a vertical line at the center of a clavicle and a right diaphragm included in the chest radiographic image. The feature detection unit 151 extracts a lung region as a chest image from the chest radiographic image by calling an object recognition algorithm stored in the memory 140. In this case, the object recognition algorithm may include an algorithm using a histogram of oriented gradients (HOG), a Haar, a local binary pattern (LBP), or the like.

The feature detection unit 151 calls an algorithm for detecting the rib cage from the chest image with respect to the extracted lung region. In this case, the algorithm for detecting the rib cage may include an algorithm for binarizing the chest image and an algorithm for removing noise to improve the form of the chest image. The feature detection unit 151 binarizes the chest image with respect to the lung region, detects the rib cage, and removes noise from the detected rib cage. In this case, the algorithm for binarizing the chest image may be, for example, an Otsu algorithm, adaptive thresholding, or the like, and the algorithm for removing noise may be an erosion algorithm, a dilation algorithm, or the like.

When the noise removal from the chest image is completed, the quality analysis unit 152 calls an inspiration-level analysis algorithm stored in the memory 140 to analyze a degree of inspiration of the patient when the chest radiographic image is captured. The quality analysis unit 152 identifies a reference point in the binarized and noise-removed chest image. The quality analysis unit 152 may calculate an average of brightness of pixels in a y-axis direction of the reference point and identify heights of peaks by applying a hysteresis algorithm. The quality analysis unit 152 may calculate distances between the peaks on the basis of the heights of the peaks and identify intervals between the ribs using the calculated distances.

The quality analysis unit 152 calls an algorithm for evaluating the inspiration-level quality of the chest image. More specifically, the quality analysis unit 152 may identify the inclusion range, the patient's posture, and the degree of inspiration from among the guidelines shown in Table 1 to evaluate the inspiration-level quality of the chest image. More specifically, the quality analysis unit 152 may check that a 1^{st} rib, a lateral costal transverse sinus, and all ribs are included in the chest image, and thus identify the inclusion range indicating whether the lungs are included in the chest image normally. The quality analysis unit 152 may check whether distances between a spinous process of a thoracic vertebra and inner ends of both clavicles are the same and positions of scapulae in the chest image, and thus identify the patient's posture indicating whether the patient's posture is aligned. The quality analysis unit 152 may check whether the contact point between the vertical line at the center of the clavicle identified in the feature detection unit 151 and the right diaphragm is lower than the lower edge of the posterior 10^{th} rib, and thus may identify the degree of inspiration.

In addition, the quality analysis unit 152 may check the number of ribs included in the chest image using the intervals between the ribs identified based on the hysteresis algorithm. In addition, the quality analysis unit 152 may check whether the backs of an anterior 6^{th} rib and the posterior 10^{th} rib are visible on a right lung. The quality analysis unit 152 may check whether posterior 9^{th} and 10^{th} ribs are visible on the diaphragm, that is, at least an 8^{th} rib is visible. Accordingly, the positions of the ribs may be identified.

The quality analysis unit 152 may check that the identified inclusion range, patient's posture, and degree of inspiration reach conditions, and when the number of ribs converges on a threshold, the backs of the anterior 6^{th} rib and the posterior 10^{th} rib are visible on the right lung, and the posterior 9^{th} and 10^{th} ribs are visible on the diaphragm, the quality analysis unit 152 may check that the identified inclusion range, patient's posture, and degree of inspiration reach the conditions. In addition, when the number of ribs is greater than or equal to a first threshold, for example, 6, and is less than or equal to a second threshold, for example, 9, it can be confirmed that the number of ribs converges on the threshold of the chest image. It is clear that the first threshold and the second threshold may be changed by an observer.

The quality analysis unit 152 may store the chest image whose quality is confirmed to be appropriate in the memory 140, and display the chest image on the display unit 130. In this case, the quality analysis unit 152 may display the called chest radiographic image, the chest image with the reference point set, the patient's identification (ID) related to the patient's name, the patient's sex, the date on which the chest radiographic image is captured, and information including the fitness of the chest image or the like. Conversely, when it is confirmed that the number of ribs does not converge on the threshold, the quality analysis unit 152 may output a message indicating that the chest radiographic image is an image not suitable for reading.

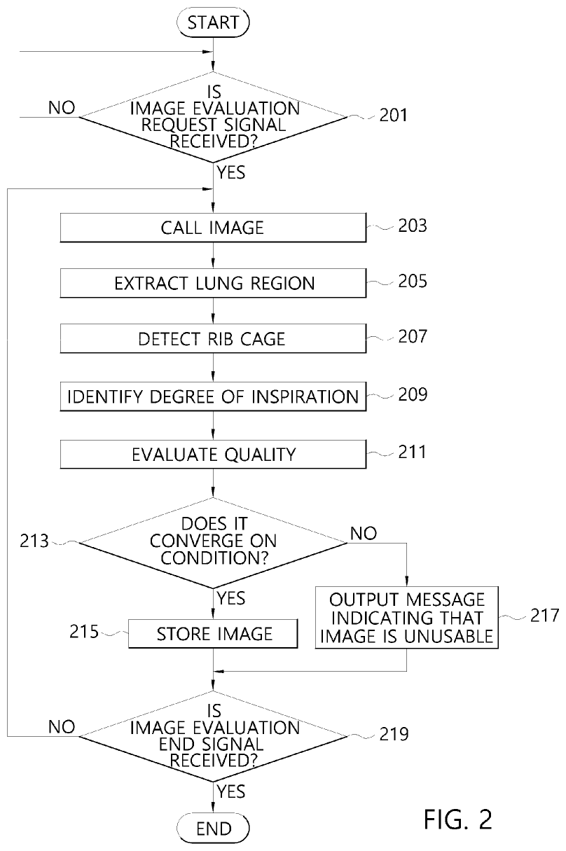

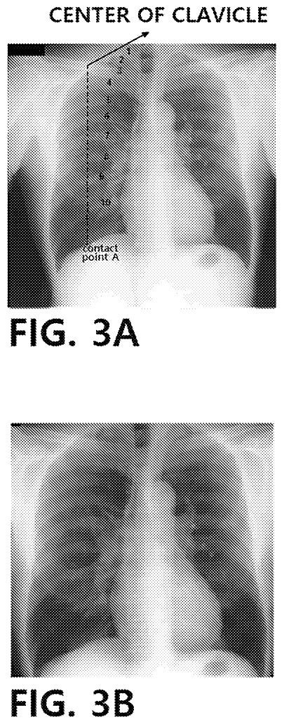

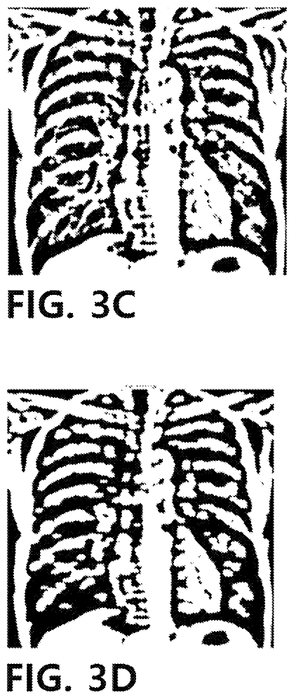

FIG. 2 is a flowchart for describing a method of evaluating inspiration-level quality of a chest radiographic image according to an embodiment of the present invention. FIGS. 3 to 5 are exemplary views of screens for describing the method of evaluating the inspiration-level quality of the chest radiographic image according to the embodiment of the present invention.

Referring to FIGS. 2 to 5, in operation 201, the feature detection unit 151 checks whether an image evaluation request signal for a chest radiographic image is received from an input unit 120. As a result of the check in operation 201, when the request signal is received, the control unit 150 performs operation 203, and when the request signal is not received, the control unit 150 waits for the reception of the request signal.

In operation 203, the feature detection unit 151 of the control unit 150 calls the chest radiographic image as shown in FIG. 3A from any one of an X-ray device (not illustrated), a server (not illustrated), and the memory 140 for image evaluation. As shown in FIG. 3A, the chest radiographic image may include 1^{st} to 10^{th} ribs. Further, the feature detection unit 151 may identify a contact point A between a vertical line at the center of a clavicle and a right diaphragm from the chest radiographic image as shown in FIG. 3A.

In operation 205, the feature detection unit 151 calls an object recognition algorithm stored in the memory 140 and extracts a lung region as a chest image from the chest radiographic image shown in FIG. 3A, as shown in FIG. 3B. In this case, the object recognition algorithm may include an algorithm using a HOG, a Haar, an LBP, or the like.

In operation 207, the feature detection unit 151 calls an algorithm for detecting a rib cage from the chest image with respect to the lung region extracted as shown in FIG. 3B. In this case, the algorithm for detecting the rib cage may include an algorithm for binarizing the chest image and an algorithm for removing noise to improve the form of the chest image. The feature detection unit 151 binarizes the chest image shown in FIG. 3C with respect to the lung region extracted as shown in FIG. 3B, detects the rib cage, and removes noise from the detected rib cage, as shown in FIG. 3D.

As shown in FIG. 3D, in the chest image after the noise removal is completed, ribs from 3^{rd} or 4^{th} to 10^{th} ribs positioned in the lung region may be identified. In this case, the algorithm for binarizing the chest image may be, for example, an Otsu algorithm, adaptive thresholding, or the like, and the algorithm for removing noise may be an erosion algorithm, a dilation algorithm, or the like.

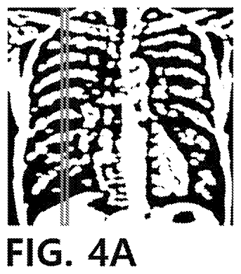

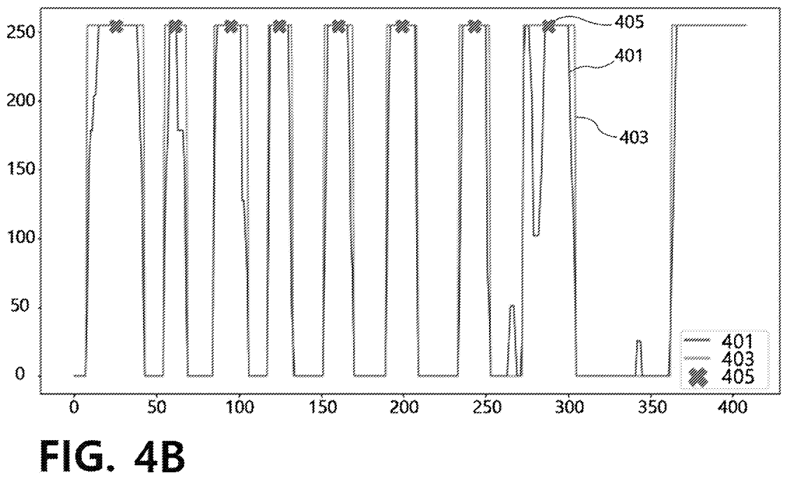

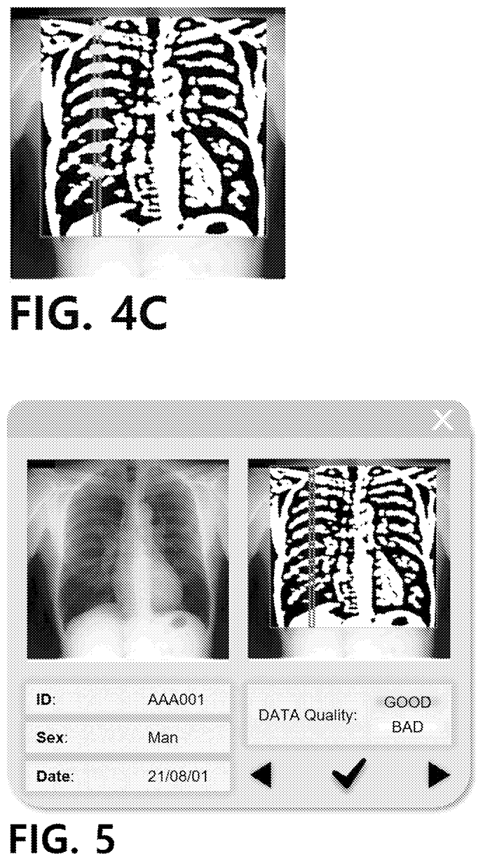

Next, in operation 209, a quality analysis unit 152 of the control unit 150 calls an inspiration-level analysis algorithm stored in the memory 140 to analyze a degree of inspiration of the patient when the chest radiographic image is captured. The quality analysis unit 152 identifies a reference point in the binarized and noise-removed chest image as shown in FIG. 3D. In this case, the quality analysis unit 152 may identify a point where the clavicle and the lungs intersect at a ¼ point on the left as a reference point, as shown in FIG. 4A.

The quality analysis unit 152 may calculate an average of brightness of pixels in a y-axis direction of the reference point and identify heights of peaks by applying a hysteresis algorithm. The quality analysis unit 152 may schematize heights of peaks as a graph as shown in FIG. 4B, calculate distances between the peaks, and identify intervals between the ribs. In FIG. 4B, reference numeral 401 is a graph based on raw data, that is, the chest radiographic image called in operation 203, and reference numeral 403 is a graph to which a hysteresis algorithm is applied. In addition, reference numeral 405 denotes peaks identified based on reference numeral 403.

Next, in operation 211, the quality analysis unit 152 calls an algorithm for evaluating inspiration-level quality of the chest image. More specifically, the quality analysis unit 152 may identify the inclusion range, the patient's posture, and the degree of inspiration from among the guidelines shown in Table 1 to evaluate the inspiration-level quality of the chest image. The quality analysis unit 152 may check that a 1^{st} rib, a lateral costal transverse sinus, and all ribs are included in the chest image, and thus identify the inclusion range indicating whether the lungs are included in the chest image normally. The quality analysis unit 152 may check whether distances between a spinous process of a thoracic vertebra and inner ends of both clavicles are the same and positions of scapulae in the chest image, and thus identify the patient's posture indicating whether the patient's posture is aligned. The quality analysis unit 152 may check whether a contact point A between a vertical line at the center of the clavicle identified in the feature detection unit 151 as shown in FIG. 3A and a right diaphragm is lower than a lower edge of the posterior 10^{th} rib, and thus identify the degree of inspiration.

In addition, the quality analysis unit 152 may check the number of ribs identified in the chest image using the intervals between the ribs identified in operation 209 as shown in FIG. 4C. The quality analysis unit 152 may check whether the backs of the anterior 6^{th} rib and the posterior 10^{th} rib are visible on a right lung. The quality analysis unit 152 may check whether posterior 9^{th} and 10^{th} ribs are visible on the diaphragm, that is, at least an 8^{th} rib is visible. Accordingly, the positions of the ribs may be identified.

In operation 213, the quality analysis unit 152 may check that the inclusion range, patient's posture, and degree of inspiration identified in operation 211 reach conditions, and when the number of ribs converges on a threshold, the backs of the anterior 6^{th} rib and the posterior 10^{th} rib are visible on the right lung, and the posterior 9^{th} and 10^{th} ribs are visible on the diaphragm, the quality analysis unit 152 may check that the identified inclusion range, patient's posture, and degree of inspiration reach the conditions, to perform operation 215. In addition, when the number of ribs is greater than or equal to a first threshold, for example, 6, and is less than or equal to a second threshold, for example, 9, it can be confirmed that the number of ribs converges on the threshold of the chest image. It is clear that the first threshold and the second threshold may be changed by an observer.

In operation 215, the quality analysis unit 152 may store the chest image whose inspiration-level quality is confirmed to be appropriate in the memory 140, and display the chest image on the display unit 130 as shown in FIG. 5. In this case, the quality analysis unit 152 may display the chest radiographic image called in operation 203, the chest image with the reference point set as shown in FIG. 4A, the patient's identification (ID) related to the patient's name, the patient's sex, the date on which the chest radiographic image is captured, and information including the fitness of the chest image or the like.

Conversely, as a result of the check in operation 213, when it is confirmed that the number of ribs does not converge on the threshold, in operation 217, the quality analysis unit 152 may output a message indicating that the chest image called in operation 203 is an image not suitable for reading.

Next, in operation 219, the control unit 150 checks whether an end signal for image evaluation is received from the input unit 120. As a result of the check in operation 219, when it is checked that the end signal is received, the control unit 150 terminates the corresponding process, and when it is not checked that the end signal is received, the control unit 150 returns to operation 203 and re-performs operations 203 to 217.

As described above, in the method and apparatus for evaluating the inspiration-level quality of the chest radiographic image according to the present invention, by determining inspiration-level quality of acquired chest radiographic images, it is possible to improve objectivity when reading images, it is possible to improve the reliability of chest radiographic images, and it is possible to reduce time and cost consumed in reading chest radiographic images.

Embodiments of the present invention disclosed in this specification and drawings are merely for providing specific examples to easily explain the technical contents of the present invention and are not intended to limit the scope of the present invention. Therefore, the scope of the present invention should be interpreted as including all changes or modifications derived on the basis of the technical spirit of the present invention in addition to the embodiments disclosed herein.

What is claimed is:

1. A method of evaluating inspiration-level quality, the method comprising:
extracting a lung region from a chest radiographic image;
detecting a rib cage from the extracted lung region;
analyzing a degree of inspiration when the chest radiographic image is captured; and
evaluating quality of the chest radiographic image.

2. The method of claim 1, wherein the evaluating of the quality includes evaluating the quality on the basis of a range in which lungs are included in the chest radiographic image identified from the extracted lung region, whether a patient's posture identified from the detected rib cage is aligned, and the degree of inspiration.

3. The method of claim 2, wherein the evaluating of the quality includes checking that a 1^{st} rib, a lateral costal transverse sinus, and all remaining ribs are included in the chest radiographic image to identify the range in which the lungs are included in the chest radiographic image.

4. The method of claim 2, wherein the evaluating of the quality includes checking whether distances between a spinous process of a thoracic vertebra and inner ends of both clavicles are the same and positions of scapulae in the chest radiographic image to check whether the patient's posture is aligned.

5. The method of claim 2, wherein the evaluating of the quality includes checking whether a contact point between a vertical line at a center of the clavicle and a right diaphragm in the chest radiographic image is lower than a lower edge of a posterior 10^{th} rib to check the degree of inspiration.

6. The method of claim 5, wherein the evaluating of the quality includes analyzing positions of the ribs and checking the number of the ribs on the basis of brightness of pixels in a y-axis direction for the ribs crossing the vertical line among the ribs identified in the chest radiographic image to evaluate the quality.

7. An apparatus for evaluating inspiration-level quality, the apparatus comprising:
an input unit configured to generate a quality evaluation request signal for a chest radiographic image; and
a control unit configured to extract a lung region from the chest radiographic image, detect a rib cage from the extracted lung region, and evaluate quality of the chest radiographic image on the basis of a degree of inspiration when the chest radiographic image is captured.

8. The apparatus of claim 7, wherein the control unit evaluates the quality on the basis of a range in which lungs are included in the chest radiographic image identified from the extracted lung region, whether a patient's posture identified from the detected rib cage is aligned, and the degree of inspiration.

9. The apparatus of claim 8, wherein the control unit checks that a 1^{st} rib, a lateral costal transverse sinus, and all ribs are included in the chest radiographic image to identify the range in which the lungs are included in the chest radiographic image.

10. The apparatus of claim 8, wherein the control unit checks whether distances between a spinous process of a thoracic vertebra and inner ends of both clavicles are the same and positions of scapulae in the chest radiographic image to check whether the patient's posture is aligned.

11. The apparatus of claim 8, wherein the control unit checks whether a contact point between a vertical line at a center of the clavicle and a right diaphragm in the chest radiographic image is lower than a lower edge of a posterior 10^{th} rib to check the degree of inspiration.

12. The apparatus of claim 11, wherein the control unit analyzes positions of the ribs and checks the number of the ribs on the basis of brightness of pixels in a y-axis direction for the ribs crossing the vertical line among the ribs identified in the chest radiographic image to evaluate the quality.

13. The apparatus of claim 7, wherein the control unit extracts the lung region from the chest radiographic image using an object recognition algorithm.

14. The apparatus of claim 7, wherein the control unit detects the rib cage from the lung region using an image binarization and noise removal algorithm.
